(19) United States
(12) Patent Application Publication
(10) Pub. No.: US 2024/0228113 A1
(43) Pub. Date: Jul. 11, 2024

(54) Inlay System

(71) Applicant: Thermo Electron LED GmbH, Langenselbold (DE)

(72) Inventor: Holger Aulbach, Kleinostheim (DE)

(21) Appl. No.: 18/404,126

(22) Filed: Jan. 4, 2024

(30) Foreign Application Priority Data
Jan. 5, 2023 (DE) 20 2023 100 040.1

(51) Int. Cl. B65D 21/02

(52) U.S. Cl. CPC B65D 21/0224

(57) ABSTRACT

An inlay system, comprising: a plurality of support structures, each of which is arranged at a different vertical level and configured to respectively support at least a portion of at least one of the plurality of containers, wherein the support structures are arranged with a horizontal offset between adjacent levels. An inlay system, comprising: a plurality of support structures, each of which is arranged at a different vertical level and configured to respectively support at least a portion of at least one of the plurality of containers, wherein a vertical spacing between a lower support structure and a higher support structure being adjacent to the lower support structure is configured so that a vertical distance V between a vertically protruding portion of at least one of the plurality of containers located on the lower support structure and the higher support structure is at most 5 cm.

### CROSS REFERENCE TO RELATED APPLICATIONS

The present application claims priority to and the benefit of German utility model application no. 20 2023 100 040.1, filed on Jan. 5, 2023. All foregoing applications are incorporated herein by reference in their entireties for any and all purposes.

### TECHNICAL FIELD

The present disclosure relates to the field of containers and to the field of laboratory supplies.

### BACKGROUND

Very generally, efficient usage of space in laboratories is highly desirable for users and/or operators of such laboratories. Consequently, the efficient use of space within equipment used in laboratories is also desirable. Generally, processing of samples stored in a respective sample container may require placement of the container within a processing device, e.g., a heating cabinet (such as a CO₂ incubator for cell culture). Overall space in such devices is limited and it is therefore desirable to use the space within such a device efficiently to advantageously allow for efficient processing of samples, e.g., cell cultures. That is, efficient usage of available space may allow to increase the throughput and thus speed-up processing and also reduce costs.

In this regard, it is known to use multi-layered sample containers comprising one or a plurality of permanently stacked trays for simultaneous processing of samples under identical conditions. Such sample containers are for example Nunc® Cell Factory® Systems by Thermo Scientific® or Corning® CellSTACK® Culture Chambers. Such sample containers comprise protruding geometries, typically located at one end portion of the top of these containers, to facilitate connection to tube inlets, tube connections, filter elements or sensors. In other words, such sample containers are typically provided with special accesses for filling, ventilation, and/or testing. These connections or attachments to the connections often protrude far beyond the outer contours of the containers. It is noted that a tube may also be referred to as a hose.

Within a processing device, e.g., an incubator, the containers are typically stored at different levels in order to efficiently use the space provided within the respective device. In other words, the containers are stacked in different levels within the respective device. For such stacking, inlay systems are typically utilised. Conventional inlay systems for such devices are designed to arrange such containers with protruding geometries directly on top of each other. However, due to the protruding geometries, such an arrangement, where the containers are positioned directly on top of each other is not very space-efficient. Further, such inlay systems do not provide space for elements at the container connections (such as tube connections or filter elements) or the vertical distance between the container levels must be large enough to provide the necessary space.

In light of the above, it is an object to overcome or at least alleviate the shortcomings and disadvantages of the prior art. More particularly, it is an object of the present invention to provide an inlay system and storage method that allows for more space-efficient storage of sample containers in processing devices such as heating cabinets, incubators or the like.

### SUMMARY

The foregoing objectives are met by the disclosed technology. In one aspect, the present disclosure provides an inlay system for storage of a plurality of containers at different vertical levels within a processing device, the inlay system comprising: a plurality of support structures, each of which is arranged at a different vertical level and configured to respectively support at least a portion of at least one of the plurality of containers, wherein the support structures are arranged with a horizontal offset (“O”) between adjacent levels.

Also provided is an inlay system for storage of a plurality of containers at different vertical levels within a processing device, the inlay system comprising: a plurality of support structures, each of which is arranged at a different vertical level and configured to respectively support at least a portion of at least one of the plurality of containers, wherein a vertical spacing between a lower support structure and a higher support structure being adjacent to the lower support structure is configured so that a vertical distance V between a vertically protruding portion of at least one of the plurality of containers located on the lower support structure and the higher support structure is at most 5 cm.

Further provided is a device for processing samples, the device comprising an inlay system for storage of a plurality of containers at different vertical levels within a processing device, the inlay system comprising a plurality of support structures, each of which is arranged at a different vertical level and configured to respectively support at least a portion of at least one of the plurality of containers, wherein the support structures are arranged with a horizontal offset between adjacent levels.

Additionally provided is a method for storing a plurality of containers at different vertical levels within a processing device, the method comprising: storing the containers with a horizontal offset between adjacent vertical levels.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1a and 1b depict a multi-layered container according to the state of the art;

FIGS. 2a to 2d depict embodiments of an inlay system comprising a horizontal offset between adjacent levels;

FIG. 3 depicts another embodiment of an inlay system comprising a horizontal offset between adjacent levels;

FIGS. 4a and 4b illustrate the improved space efficiency of an inlay system according to the present invention compared to the state of the art;

FIG. 5 depicts a further embodiment of an inlay system comprising a horizontal offset between adjacent levels, wherein the support structures directly support only a portion of a respective container;

FIG. 6 illustrates an inlay system with support structures comprising stiffening elements; and

FIG. 7 illustrates an inlay system according to the present invention within a processing device.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4.

Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

In a first aspect, the present invention relates to an inlay system for storage of a plurality of containers at different vertical levels within a processing device, the inlay system comprising a plurality of support structures, each of which is arranged at a different vertical level and configured to respectively support at least a portion of at least one of the plurality of containers. In other words, the present invention is generally related to a storage system for storing a plurality of containers at different levels within a processing device, i.e. in an interior volume of the processing device such as a chamber.

The term processing device may generally relate to any device for processing, treatment or even storage of samples in a device that may for example provide controlled conditions such as a controlled temperature, humidity, CO₂-Level, or the like.

It will be understood that each of the support structures may support one or more of the plurality of containers, and that each container is only supported by a single support structure since each container may only be placed at a single level.

A vertical spacing between a lower support structure and a higher support structure being adjacent to the lower support structure may be configured so that a vertical distance V between a vertically protruding portion of at least one of the plurality of containers located on the lower support structure and the higher support structure is at most 5 cm, preferably at most 2.5 cm, more preferably at most 1 cm. That is, the vertical spacing between adjacent support structures may be such that the vertical distance between the higher support structure and the top of a vertically protruding portion (i.e. the upwards end of the vertically protruding portion) of a container stored on the lower support structure is limited to at most 5 cm, preferably at most 2.5 cm, more preferably at most 1 cm. It will be understood that vertical spacing refers to the vertical distance between the protruding portion and the higher support structure. Furthermore, it will be understood that the above merely relates to an upper limit for the vertical spacing and that the vertical distance may for example also be 0, in particular there may be an overlap, i.e. no vertical spacing.

Furthermore, it will be understood that generally the vertical distance between different pairs of adjacent support structures may vary, for example due to different sizes and particular heights of containers to be stored in the inlay system.

The vertical spacing between a lower support structure and a higher support structure being adjacent to the lower support structure may be configured so that a vertically protruding portion of at least one of the plurality of containers located on the lower support structure vertically overlaps and/or extends beyond the higher support structure. That is, the vertical spacing between adjacent support structures may be such that the vertically protruding portion of a container stored on the lower support structure at least vertically overlaps with at least a portion of the higher support structure.

It will be understood that to “vertically overlap” is to be construed as to having an overlap in the vertical extension. In other words, two objects vertically overlap when they can both be cut by a single horizontal line. Similarly, “horizontally overlap” is to be construed as to having an overlap in the horizontal extension. In other words, two objects horizontally overlap when they can both be cut by a single vertical line.

It will be understood that the level below and above a current level are both referred to as “an adjacent level”. That is, an adjacent level may denote the level directly below and/or above the current level.

In some embodiments, the support structures may be arranged with a horizontal offset between adjacent levels. It will be understood that the horizontal offset may be different for different pairs of adjacent levels. Furthermore, it will be understood that the horizontal offset may only be present at one end of the support structure, e.g., in cases where the support structures have different lengths. For example, support structures may be arranged within a processing device, such that they have a horizontal offset at their respective front ends, i.e. the ends closest to an opening for accessing an inner volume of the processing device in which the inlay system is installed, while each of the support structures ends at the same point at the respective back end opposing the front end. Further, the horizontal offset may be configured so that the vertical distance V between a vertically protruding portion of at least one of the plurality of containers and the support structure of the adjacent level directly above the level on which the at least one container is located is at most 5 cm, preferably at most 2.5 cm, more preferably at most 1 cm. Additionally or alternatively, the horizontal offset may be configured so that a vertically protruding portion of at least one of the plurality of containers vertically overlaps and/or extends beyond the support structure of the adjacent level directly above the level on which the at least one container is located.

The horizontal offset may be such that neither the support structure adjacent in the direction of the vertically protruding portion of at least one of the plurality of containers nor a container stored upon said adjacent support structure horizontally overlaps with said protruding portion. In other words, the horizontal offset between adjacent levels may be such that for a container stored in the inlay system neither the support structure adjacent in the direction of the vertically protruding portion of said container nor another container stored upon said support structure overlap in their horizontal extension with said protruding portion. This may advantageously allow to access protruding portions of stored containers without the need for an extensive vertical spacing, such that vertical space in a processing device may be used in a more space-efficient way. The horizontal offset between adjacent levels may be in the same direction for each level. Alternatively, the horizontal offset between adjacent levels may alternate in opposite directions.

Each of the plurality of support structures may be configured to fully support at least one container. Alternatively, each of the plurality of support structures may be configured to directly support only a portion of at least one container. Further, each of the plurality of support structures may be configured to directly support at most 95%, preferably at most 90% of a footprint of the at least one container.

In some embodiments, each of the plurality of support structures may be configured to directly support at least 50%, preferably at least 60% more preferably at least 75% of a footprint of the at least one container.

Each of the plurality of support structures may be made of metal. For example, each of the plurality of support structures may be made of a sheet metal or a wire mesh. The sheet metal may be a bent sheet metal. The sheet metal or wire mesh may be made of at least one of stainless steel, coated steel and copper. The coated steel may be galvanized or nickel-plated.

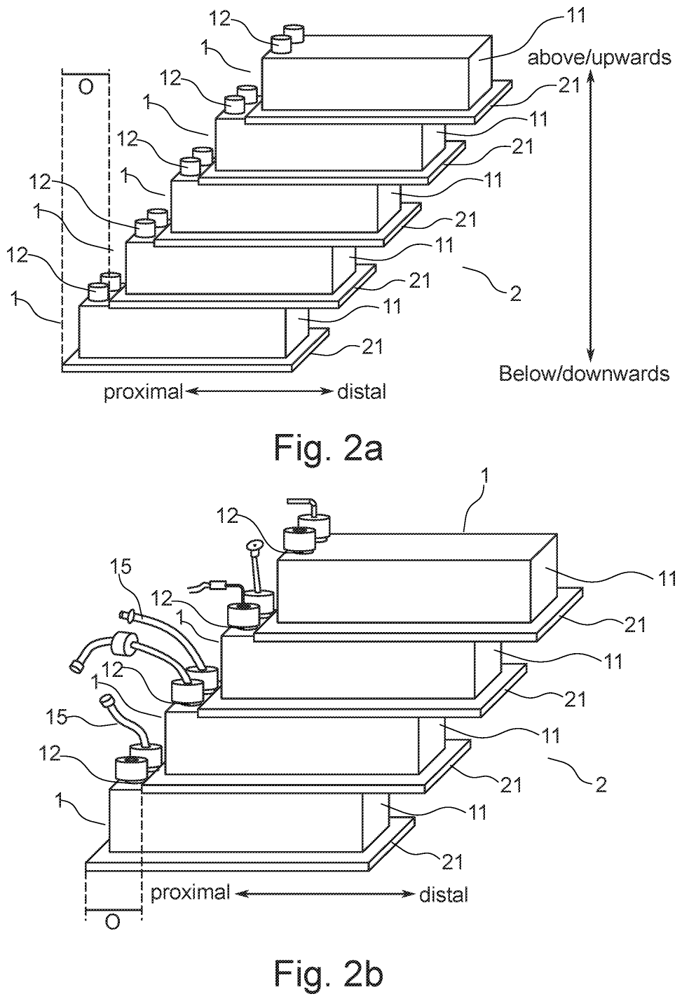

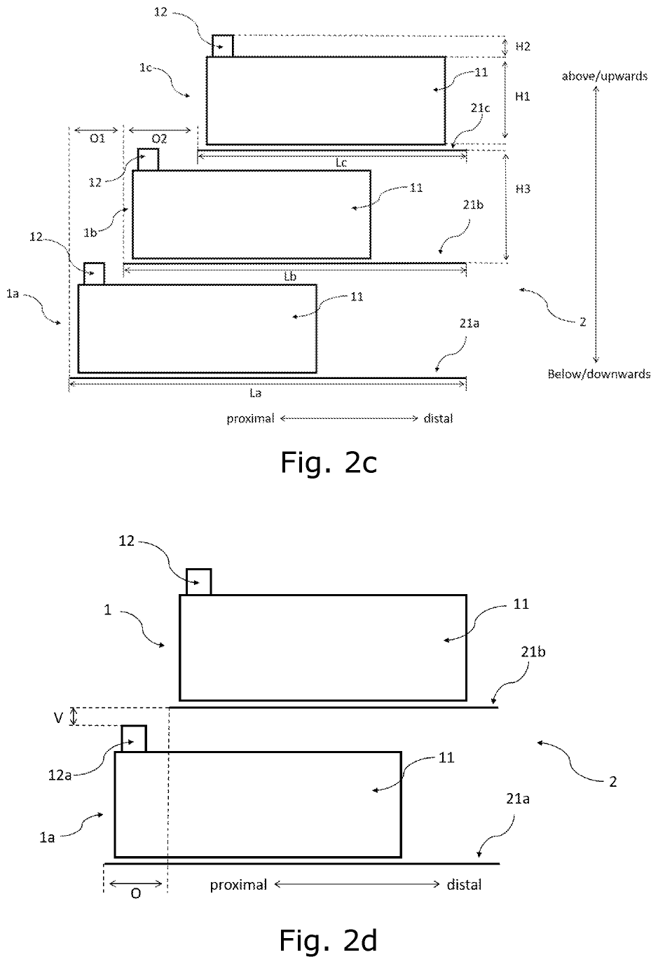

Each of the plurality of support structures may comprise an identical support structure length. It will be understood that support structure length denotes the extension of the support structure in horizontal direction, particularly in a proximal to distal direction (cf. FIG. 2c below). In other words, the support structure length may denote the extension of a respective support structure in a direction running from an opening of the processing device in which the inlay system is installed to a backside of the inner volume, i.e. the side of the inner volume lying opposite to the opening through which the inner volume of the processing device may be accessed. If the inlay system comprises a horizontal offset, the direction corresponds to the direction in which the support structures are horizontally offset. Alternatively, the plurality of support structures may comprise support structures comprising different support structure lengths. In other words, at least some of the support structures may differ in length. In some embodiments, each of the plurality of support structures may comprise a different support structure length.

At least one of the support structures may comprise at least one stiffening element. Such a stiffening element may advantageously provide additional stiffening for the respective support structure. For example, a stiffening element may reduce and preferably prevent the support structure bending down owing to the weight of containers stored thereon. Further, the at least one stiffening element may comprise at least one of a bend, an offset, a hem, a hole, a slot, a seam, a tab, a notch, a flange, an emboss, a dimple and/or a rib.

The at least one of the at least one stiffening element may be provided through bending, flanging, hemming, and/or embossing. Generally, the at least one stiffening element may comprise a bracket, or a plate, such as a sheet metal flap.

An arrangement and/or a vertical extension of any stiffening element may be such that it does not obstruct removing a container on a level below the support structure. The vertical spacing between the lower support structure and the higher support structure may be configured so that that a vertical distance V between the vertically protruding portion of at least one of the plurality of containers located on the lower support structure and at least one of the at least one stiffening element of the higher support structure is at most 5 cm, preferably at most 2.5 cm, more preferably at most 1 cm.

The vertical spacing between the lower support structure and the higher support structure may be configured so that a vertically protruding portion of at least one of the plurality of containers located on the lower support structure vertically overlaps and/or extends beyond at least one of the at least one stiffening element of the higher support structure.

The system may further comprise at least one vertical strut configured to provide additional support to at least one of the support structures. That is, in addition to the at least one stiffening element, the system may comprise at least one vertical strut which may provide an additional support to the support structures and thus for example also contribute to reducing and/or avoiding a bending down of the support structure under load. The at least one vertical strut may vertically connect a respective support structure to another support structure and/or an inner surface of the processing device. For example, the vertical strut may connect the respective support structure to the floor of an inner volume of the processing device or another mounting point of the processing device, e.g., located at a surface defining the inner volume of the processing device. The vertical strut may be located such that it does not obstruct insertion and/or removal of containers. Advantageously the vertical strut may be configured for providing support to a central region of the support structure, e.g., a region located centrally between mounting points of the support structure. The system may comprise at least one vertical strut for each support structure.

Each of the plurality of containers may comprise a main portion and at least one of the plurality of containers may comprise at least one protruding portion that extends vertically beyond the main portion of the container. It will be understood that the main portion defines the overall contour of the container, while one or more protruding portions may extend beyond the main portion, and thus beyond the general contour of the container. Furthermore, it will be understood that the “protruding portion” may also be referred to as “vertically protruding portion”. In some embodiments, each of the plurality of containers may comprise at least one protruding portion that extends vertically beyond the main portion of the container.

The main portion of at least one container may resemble a cube or cuboid shape. At least one protruding portion of at least one of the plurality of containers may be vertically distanced from the support structure of an adjacent level higher than the level supporting the at least one of the plurality of containers by at most 5 cm, preferably at most 2.5 cm, more preferably at most 1 cm when stored in the inlay system. Additionally or alternatively, at least one protruding portion of at least one of the plurality of containers may vertically overlap and/or extend beyond the support structure of an adjacent level higher than the level supporting the at least one of the plurality of containers when stored in the inlay system.

The combination of vertical spacing and horizontal offset between adjacent levels may be such that a vertical distance between the vertically protruding portion of at least one of the plurality of containers located on the lower support structure and the higher support structure is at most 5 cm, preferably at most 2.5 cm, more preferably at most 1 cm. Additionally or alternatively, the combination of vertical spacing and horizontal offset between adjacent levels may be such that at least one protruding portion of at least one container vertically overlaps and/or extends beyond the support structure of an adjacent level higher than the level supporting the at least one of the plurality of containers.

The at least one protruding portion may provide an access to an inner volume of the container. In other words, the container may comprise an inner volume which may be accessible through the at least one portions comprised by said container. The access may be for at least one of at least partially filling of the container, ventilation of the container, inoculation, feeding, harvesting and/or testing of a sample within the container. In particular, the access may be configured for providing and/or extracting a fluid such as a gas or liquid from or respectively to the inner volume of the container.

The protruding portion may provide a connector for connection to at least one or a plurality of a cap, a vent cap, a filter assembly, a tubing, a fitting, a connector, an adaptor cap, a port cover, a vent filter, a tube inlet, a tube connector, a filter element and/or a sensor. That is, the protruding portion may for example comprise a thread or another form of fitting for connecting at least one of the above-mentioned elements, particularly for providing a fluid-tight connection to one of the above-mentioned elements. Thereby the protruding portion may provide a connector allowing for easy access to the inner volume of the container and allow for connecting desired elements thereto, e.g., elements mentioned above. In other words, the protruding portion may provide a port allowing for easy access to the inner volume of the container, wherein the port is configured for connection to external elements such as the ones mentioned above.

The at least one protruding portion may be located in a proximal section of the container that is more proximal than the remaining part of the container in a horizontal direction. The proximal section may refer to a front (or back) end section of the container. It will be understood that the proximal section may comprise part of the main portion.

The at least one protruding portion of the at least one container may comprise a protruding portion height H2. In other words, the at least one protruding portion may comprise a vertical extension which may be referred to as protruding portion height H2. It will be understood that different protruding portions may comprise a different protruding portion height H2. For example, one container may comprise two protruding portions each of which comprises a different protruding portion height.

At least one of the plurality of containers may further comprise a rim vertically extending from the main portion of said container and running around at least a portion of a circumference of the main portion. In other words, a top surface of the main portion, e.g., the surface from which the protruding portion extends, may at least partially be surrounded by a vertically extending rim. The rim may comprise a rim height, wherein for a container comprising at least one protruding portion and a rim, the rim height may be smaller than the protruding portion height H2. That is, for a container comprising a rim and at least one protruding portion, both vertically extending from the main portion in the same direction, the protruding portion may extend further than the rim, i.e. it may comprise a greater height. In some embodiments, the rim may not extend into the proximal section of the container. In other words, the rim may be limited to a part of the main body that does not comprise the proximal section.

In some embodiments, any stiffening element extending vertically downwards may be located distal of the respective proximal section of a container stored in the level below the respective stiffening element. The term “downwards” denotes the vertical direction away from a support structure in the direction opposite to the side on which the container is stored. Additionally or alternatively, any stiffening element extending vertically downwards may be located distal of the respective protruding portion of a container stored in the level below the respective stiffening element.

The plurality of containers may be comprised by the inlay system. The plurality of containers may be configured to store and/or process a sample. For example, the plurality of containers may be configured for cultivating cells. At least one of the plurality of containers may be a multi-layer container. A multi-layer container may provide a plurality of layers with similar, preferably identical conditions, e.g., through stacked trays. For example, at least one of the plurality containers comprises a plurality of permanently stacked trays with at least one common fluid connection within the container. It will be understood that the number of layers/trays of a container may differ between containers of the plurality of containers.

The main portion may comprise main portion height H1. In other words, a vertical extension of the main portion may be denoted as main portion height H1. For at least one container comprising a protruding portion and being stored on a lower support structure a vertical distance H3 between the lower support structure and a higher support structure being adjacent to the lower support structure may be chosen such that H1<H3<(H1+H2).

The main portion height H1 may be in the range of 0.5 cm to 100 cm, preferably 1 cm to 80 cm, more preferably 1.5 cm to 75 cm. The protruding portion height H2 may be in the range of 0.5 cm to 10 cm, preferably 0.5 cm to 5 cm, more preferably 1 cm to 4 cm.

In some embodiments, the proximal section may comprise a proximal section width W and wherein the horizontal offset is greater than the proximal section width W. The proximal section width W may be in the range of 2 cm to 5 cm

The processing device may be one of a heated and/or refrigerated incubator, an environmental chamber, a heating cabinet or a cooling cabinet. Very generally, the processing device may comprise a chamber defining an inner volume which may be accessed through an opening, e.g., via a door. Furthermore, the processing device may be configured to control at least one parameter within the chamber, e.g., temperature, humidity, gas composition, etc.

The horizontal offset O may be in the range of 2 to 10 cm, preferably 3 to 8 cm, more preferably 4 to 6 cm. Again, it will be understood that the horizontal offset may vary for different pairs of adjacent support structures. Thus, in some embodiments the horizontal offset O may be different between different adjacent levels. Alternatively, the horizontal offset O may be identical for all adjacent levels.

A higher support structure being adjacent to a lower support structure may be configured such that a vertically protruding portion of at least one of the plurality of containers located on the lower support structure does not horizontally overlap with the respective higher support structure.

Each of the plurality of support structures may be configured to respectively support at least a portion of a plurality of the plurality of containers. In some embodiments, the proximal section may not directly be supported by the support structure.

In a further aspect the present invention relates to a device for processing samples, the device comprising an inlay system for storage of a plurality of containers at different vertical levels within the processing device as described above. That is, the processing device may comprise the above-described inlay system, e.g., said inlay system may be installed within an inner volume of the processing device, such as a chamber of the processing device.

The term processing samples may generally relate to any processing, treatment or even storage of samples in a device that may for example provide required conditions such as a controlled temperature, humidity, CO₂-Level, or the like.

The support structures of the inlay system may be arranged with a horizontal offset between adjacent levels. This may advantageously allow to store containers within the processing device in a more space-efficient manner compared to utilizing known inlay systems not comprising a horizontal offset. Furthermore, the device may be configured to control at least one or a plurality of heat, temperature, humidity, CO₂-Level, and/or O₂-level within the processing device and/or containers stored therein. That is, the processing device may comprise an inner volume, e.g., a chamber, within which the inlay system is installed and may be configured to control at least one or a plurality of heat, temperature, humidity, CO₂-level, and/or O₂-level within said inner volume/chamber and/or containers stored therein.

The device may be at least one of an incubator, a cooling cabinet, a heating cabinet, and/or an environmental chamber.

In another aspect, the present invention relates to a method for storing a plurality of containers at different vertical levels within a processing device. In a first embodiment, the method comprises storing the containers such that a vertical distance between a vertically protruding portion of at least one of the plurality of containers and a container at an adjacent level being higher than the level on which the at least one of the plurality of containers is stored is at most 5 cm, preferably at most 2.5 cm, more preferably at most 1 cm.

In a second, alternative embodiment, the method comprises storing the containers such that a vertically protruding portion of at least one of the plurality of containers vertically overlaps and/or extends beyond a container at an adjacent level being higher than the level on which the at least one of the plurality of containers is stored.

Furthermore, the features of the first and second embodiment may be combined.

In a third embodiment, the method comprises storing the containers with a horizontal offset between adjacent vertical levels.

Furthermore, the features of the third embodiment may be combined with the features of the first and second embodiment.

In the following additional method features will be described that may be combined with the above first, second and/or third embodiment.

The method may comprise storing the plurality of containers in an inlay system comprising a plurality of support structures, each of which is configured to support at least a portion of at least one container. The inlay system may be an inlay system as described above.

The method may comprise storing the containers such that a vertical distance V between a vertically protruding portion of at least one of the plurality of containers and the support structure of an adjacent level being higher than the level supporting the at least one of the plurality of containers is at most 5 cm, preferably at most 2.5 cm, more preferably at most 1 cm. Again, it will be understood that the vertical distance may for example also be 0, in particular there may be a vertical overlap between the protruding portion and the support structure of said adjacent level. Additionally or alternatively, the method may comprise storing the containers such that a vertically protruding portion of at least one of the plurality of containers vertically overlaps and/or extends beyond the support structure of an adjacent level being higher than the level supporting the at least one of the plurality of containers.

The method may comprise storing the containers such that neither the support structure adjacent in the direction of the vertically protruding portion of at least one of the plurality of containers nor a container stored upon said adjacent support structure horizontally overlaps with said protruding portion.

The method may comprise storing each of the plurality of containers on a support structure of the inlay system.

The method may comprise installing the inlay system in a processing device such that there is a horizontal offset between support structures of adjacent levels. Generally, the horizontal offset between adjacent levels may be in the same direction for each level for some embodiments, while for other embodiments the horizontal offset between adjacent levels may alternate in opposite directions.

The method may comprise removing a container from one level without removing the container of the above level.

The method may comprise providing a fluid connection to at least one of the plurality of containers, wherein the fluid connection vertically overlaps and/or extends beyond a container at an adjacent level. In particular, the fluid connection may be provided to a container via a protruding portion of said container. Additionally or alternatively, the method may comprise providing a fluid connection to at least one of the plurality of containers, wherein the fluid connection vertically overlaps and/or extends beyond the support structure of an adjacent level being higher than the level supporting the at least one of the plurality of containers.

The method may further comprise at least partially filling the container with a fluid. Further, the method may comprise ventilating the container.

The method may further comprise one or a plurality of inoculating, feeding, harvesting and/or testing of a sample within the container. In some embodiments, the method may comprise cultivating cells within the plurality of containers

The processing device in which the plurality of containers is stored may be a device according to any of the preceding device embodiments.

In another aspect, the present invention relates to a use of the inlay system as described above for storing a plurality of containers at different vertical levels within a processing device. The processing device may be a device as described above. Additionally or alternatively, the use may be for performing the method as described above.

Below, reference will be made to inlay system embodiments. These embodiments are abbreviated by the letter “S” followed by a number. Whenever reference is herein made to “system embodiments”, these embodiments are meant.

S1. An inlay system for storage of a plurality of containers at different vertical levels within a processing device, the inlay system comprising a plurality of support structures, each of which is arranged at a different vertical level and configured to respectively support at least a portion of at least one of the plurality of containers.

The term processing device may generally relate to any device for processing, treatment or even storage of samples in a device that may for example provide controlled conditions such as a controlled temperature, humidity, CO₂-Level, or the like.

It will be understood that each of the support structures may support one or more of the plurality of containers, and that each container is only supported by a single support structure since each container may only be placed at a single level.

S2. The inlay system according to the preceding system embodiment, wherein a vertical spacing between a lower support structure and a higher support structure being adjacent to the lower support structure is configured so that a vertical distance V between a vertically protruding portion of at least one of the plurality of containers located on the lower support structure and the higher support structure is at most 5 cm, preferably at most 2.5 cm, more preferably at most 1 cm.

S3. The inlay system according to any of the preceding system embodiments, wherein the vertical spacing between a lower support structure and a higher support structure being adjacent to the lower support structure is configured so that a vertically protruding portion of at least one of the plurality of containers located on the lower support structure vertically overlaps and/or extends beyond the higher support structure.

It will be understood that the level below and above a current level are both referred to as “an adjacent level”. That is, an adjacent level may denote the level directly below and/or above the current level.

S4. The inlay system according to any of the preceding system embodiments, wherein the support structures are arranged with a horizontal offset between adjacent levels.

S5. The inlay system according to the preceding system embodiment, wherein the horizontal offset is configured so that the vertical distance V between a vertically protruding portion of at least one of the plurality of containers and the support structure of the adjacent level directly above the level on which the at least one container is located is at most 5 cm, preferably at most 2.5 cm, more preferably at most 1 cm.

S6. The inlay system according to any of the 2 preceding system embodiments, wherein the horizontal offset is configured so that a vertically protruding portion of at least one of the plurality of containers vertically overlaps and/or extends beyond the support structure of the adjacent level directly above the level on which the at least one container is located.

S7. The inlay system according to any of the 3 preceding system embodiments, wherein the horizontal offset is such that neither the support structure adjacent in the direction of the vertically protruding portion of at least one of the plurality of containers nor a container stored upon said adjacent support structure horizontally overlaps with said protruding portion.

S8. The inlay system according to any of the 4 preceding system embodiments, wherein the horizontal offset between adjacent levels is in the same direction for each level.

S9. The inlay system according to any of the 5 preceding system embodiments excluding the features of S8, wherein the horizontal offset between adjacent levels alternates in opposite directions.

S10. The inlay system according to any of the preceding system embodiments, wherein each of the plurality of support structures is configured to fully support at least one container.

S11. The inlay system according to any of the preceding system embodiments excluding the features of S10, wherein each of the plurality of support structures is configured to directly support only a portion of at least one container.

S12. The inlay system according to the preceding system embodiment, wherein each of the plurality of support structures is configured to directly support at most 95%, preferably at most 90% of a footprint of the at least one container.

S13. The inlay system according to any preceding system embodiment, wherein each of the plurality of support structures is configured to directly support at least 50%, preferably at least 60% more preferably at least 75% of a footprint of the at least one container.

S14. The inlay system according to any of the preceding system embodiment, wherein each of the plurality of support structures is made of metal.

S15. The inlay system according to the preceding system embodiment, wherein each of the plurality of support structures is made of a sheet metal or a wire mesh.

S16. The inlay system according to the preceding system embodiment, wherein the sheet metal is a bent sheet metal.

S17. The inlay system according to any of the 2 preceding system embodiments, wherein the sheet metal or wire mesh is made of at least one of stainless steel, coated steel and copper.

S18. The inlay system according to the preceding system embodiment, wherein the coated steel is galvanized or nickel-plated.

S19. The inlay system according to any of the preceding system embodiments, wherein each of the plurality of support structures comprises an identical support structure length.

S20. The inlay system according to any of the preceding system embodiments without the features of S19, wherein the plurality of support structures comprises support structures comprising different support structure lengths.

S21. The inlay system according to the preceding system embodiment, wherein each of the plurality of support structures comprises a different support structure length.

S22. The inlay system according to any of the preceding system embodiments, wherein at least one of the support structures comprises at least one stiffening element.

S23. The inlay system according to the preceding system embodiment, wherein the at least one stiffening element comprises at least one of a bend, an offset, a hem, a hole, a slot, a seam, a tab, a notch, a flange, an emboss, a dimple and/or a rib.

S24. The inlay system according to any of the 2 preceding system embodiments, wherein at least one of the at least one stiffening element is provided through bending, flanging, hemming, and/or embossing.

S25. The inlay system according to any of the 3 preceding system embodiments, wherein the at least one stiffening element comprises a bracket, or a plate, such as a sheet metal flap.

S26. The inlay system according to any of the 4 preceding system embodiments, wherein an arrangement and/or a vertical extension of any stiffening element is such that it does not obstruct removing a container on a level below the support structure.

S27. The inlay system according to the preceding system embodiment and with the features of S2, wherein the vertical spacing between the lower support structure and the higher support structure is configured so that a vertical distance V between the vertically protruding portion of at least one of the plurality of containers located on the lower support structure and at least one of the at least one stiffening element of the higher support structure is at most 5 cm, preferably at most 2.5 cm, more preferably at most 1 cm.

S28. The inlay system according to any of the 2 preceding system embodiments and with the features of S2 or S3, wherein the vertical spacing between the lower support structure and the higher support structure is configured so that a vertically protruding portion of at least one of the plurality of containers located on the lower support structure vertically overlaps and/or extends beyond at least one of the at least one stiffening element of the higher support structure.

S29. The inlay system according to any of the preceding system embodiments, wherein the system further comprises at least one vertical strut configured to provide additional support to at least one of the support structures.

S30. The inlay system according to the preceding system embodiment, wherein the at least one vertical strut vertically connects a respective support structure to another support structure and/or an inner surface of the processing device.

S31. The inlay system according to any of the 2 preceding system embodiments, wherein the system comprises at least one vertical strut for each support structure.

S32. The inlay system according to any of the preceding system embodiments, wherein each of the plurality of containers comprises a main portion and at least one of the plurality of containers comprises at least one protruding portion that extends vertically beyond the main portion of the container.

It will be understood that the main portion defines the overall contour of the container, while one or more protruding portions may extend beyond the main portion, and thus beyond the general contour of the container.

Furthermore, it will be understood that the “protruding portion” may also be referred to as “vertically protruding portion”.

S33. The inlay system according to the preceding system embodiment, wherein each of the plurality of containers comprises at least one protruding portion that extends vertically beyond the main portion of the container.

S34. The inlay system according to any of the 2 preceding system embodiments, wherein the main portion of at least one container resembles a cube or cuboid shape.

S35. The inlay system according to any of the 3 preceding system embodiments, wherein at least one protruding portion of at least one of the plurality of containers is vertically distanced from the support structure of an adjacent level higher than the level supporting the at least one of the plurality of containers by at most 5 cm, preferably at most 2.5 cm, more preferably at most 1 cm when stored in the inlay system.

S36. The inlay system according to any of the 4 preceding system embodiments, wherein at least one protruding portion of at least one of the plurality of containers vertically overlaps and/or extends beyond the support structure of an adjacent level higher than the level supporting the at least one of the plurality of containers when stored in the inlay system.

S37. The inlay system according to any of the 5 preceding system embodiments and with the features of S2 or S3, and S4, wherein the combination of vertical spacing and horizontal offset between adjacent levels is such that a vertical distance between the vertically protruding portion of at least one of the plurality of containers located on the lower support structure and the higher support structure is at most 5 cm, preferably at most 2.5 cm, more preferably at most 1 cm.

S38. The inlay system according to any of the 6 preceding system embodiments and with the features of S2 or S3, and S4, wherein the combination of vertical spacing and horizontal offset between adjacent levels is such that at least one protruding portion of at least one container vertically overlaps and/or extends beyond the support structure of an adjacent level higher than the level supporting the at least one of the plurality of containers.

S39. The inlay system according to any of the 7 preceding system embodiments, wherein the at least one protruding portion provides an access to an inner volume of the container.

S40. The inlay system according to the preceding system embodiment, wherein the access is for at least one of at least partially filling of the container, ventilation of the container, inoculation, feeding, harvesting and/or testing of a sample within the container.

S41. The inlay system according to any of the 9 preceding system embodiments, wherein the protruding portion provides a connector for connection to at least one or a plurality of a cap, a vent cap, a filter assembly, a tubing, a fitting, a connector, an adaptor cap, a port cover, a vent filter, a tube inlet, a tube connector, a filter element and/or a sensor.

S42. The inlay system according to any of the 10 preceding system embodiments, wherein the at least one protruding portion is located in a proximal section of the container that is more proximal than the remaining part of the container in a horizontal direction.

S43. The inlay system according to any of the preceding system embodiments and with the features of S32, wherein the at least one protruding portion of the at least one container comprises a protruding portion height H2.

S44. The inlay system according to any of the preceding system embodiments and with the features of S32, wherein at least one of the plurality of containers further comprises a rim vertically extending from the main portion of said container and running around at least a portion of a circumference of the main portion.

S45. The inlay system according to the preceding system embodiment and with the features of S43, wherein the rim comprises a rim height and wherein for a container comprising at least one protruding portion and a rim, the rim height is smaller than the protruding portion height H2.

S46. The inlay system according to any of the 2 preceding system embodiments and with the features of S42, wherein the rim does not extend into the proximal section of the container.

S47. The inlay system according to any of the preceding system embodiments and with the features of S22 and S42, wherein any stiffening element extending vertically downwards is located distal of the respective proximal section of a container stored in the level below the respective stiffening element.

The term “downwards” denotes the vertical direction away from a support structure in the direction opposite to the side on which the container is stored.

S48. The inlay system according to any of the preceding system embodiments and with the features of S22, wherein any stiffening element extending vertically downwards is located distal of the respective protruding portion of a container stored in the level below the respective stiffening element.

S49. The inlay system according to any of the preceding system embodiments, wherein the plurality of containers is comprised by the inlay system.

S50. The inlay system according to the preceding system embodiment, wherein the plurality of containers is configured to store and/or process a sample.

S51. The inlay system according to any of the 2 preceding system embodiments, wherein at least one of the plurality of containers is a multi-layer container.

S52. The inlay system according to any of the 3 preceding system embodiments, wherein at least one of the plurality containers comprises a plurality of permanently stacked trays with at least one common fluid connection within the container.

It will be understood that the number of layers/trays of a container may differ between containers of the plurality of containers.

S53. The inlay system according to any of the preceding system embodiments and with the features of S32, wherein the main portion comprises main portion height H1.

S54. The inlay system according to the preceding system embodiment and with the features of S43, wherein for at least one container comprising a protruding portion and being stored on a lower support structure a vertical distance H3 between the lower support structure and a higher support structure being adjacent to the lower support structure is chosen such that H1<H3<(H1+H2).

S55. The inlay system according to any of the 2 the preceding system embodiments, wherein the main portion height H1 is in the range of 0.5 cm to 100 cm, preferably 1 cm to 80 cm, more preferably 1.5 cm to 75 cm.

S56. The inlay system according to any of the preceding system embodiments and with the features of S43, wherein the protruding portion height H2 is in the range of 0.5 cm to 10 cm, preferably 0.5 cm to 5 cm, more preferably 1 cm to 4 cm.

S57. The inlay system according to any of the preceding system embodiments and with the features of S4 and S42, wherein the proximal section comprises a proximal section width W and wherein the horizontal offset is greater than the proximal section width W.

S58. The inlay system according to the preceding system embodiment, wherein the proximal section width W is in the range of 2 to 5 cm.

S59. The inlay system according to any of the preceding system embodiments, wherein the processing device is one of a heated and/or refrigerated incubator, an environmental chamber, a heating cabinet or a cooling cabinet.

S60. The inlay system according to any of the preceding system embodiments and with the features of S4, wherein the horizontal offset O is in the range of 2 to 10 cm, preferably 3 to 8 cm, more preferably 4 to 6 cm.

S61. The inlay system according to any of the preceding system embodiments and with the features of S4, wherein the horizontal offset O is different between different adjacent levels.

S62. The inlay system according to any of the preceding system embodiments with the features of S4 and without the features of S61, wherein the horizontal offset O is identical for all adjacent levels.

S63. The inlay system according to any of the preceding system embodiments, wherein a higher support structure being adjacent to a lower support structure is configured such that a vertically protruding portion of at least one of the plurality of containers located on the lower support structure does not horizontally overlap with the respective higher support structure.

S64. The inlay system according to any of the preceding system embodiments, wherein each of the plurality of support structures is configured to respectively support at least a portion of a plurality of the plurality of containers.

S65. The inlay system according to any of the preceding system embodiments and with the features of S42 and S11, wherein the proximal section is not directly supported by the support structure.

Below, reference will be made to device embodiments. These embodiments are abbreviated by the letter “D” followed by a number. Whenever reference is herein made to “device embodiments”, these embodiments are meant.

D1. A device for processing samples, the device comprising an inlay system for storage of a plurality of containers at different vertical levels within the processing device according to any of the preceding system embodiments.

D2. The device according to any of the preceding device embodiments, wherein the support structures of the inlay system are arranged with a horizontal offset between adjacent levels.

D3. The device according to any of the preceding device embodiments, wherein the device is configured to control at least one or a plurality of temperature, humidity, CO₂-Level, and/or O₂-Level within the processing device and/or containers stored therein.

D4. The device according to any of the preceding device embodiments, wherein the device is an incubator.

D5. The device according to any of the preceding device embodiments, wherein the device is a cooling cabinet.

D6. The device according to any of the preceding device embodiments, wherein the device is a heating cabinet.

D7. The device according to any of the preceding device embodiments, wherein the device is an environmental chamber.

The term processing samples may generally relate to any processing, treatment or even storage of samples in a device that may for example provide required conditions such as a controlled temperature, humidity, CO₂-Level, or the like.

Below, reference will be made to method embodiments. These embodiments are abbreviated by the letter “M” followed by a number. Whenever reference is herein made to “method embodiments”, these embodiments are meant.

M1. Method for storing a plurality of containers at different vertical levels within a processing device, the method comprising

storing the containers such that a vertical distance between a vertically protruding portion of at least one of the plurality of containers and a container at an adjacent level being higher than the level on which the at least one of the plurality of containers is stored is at most 5 cm, preferably at most 2.5 cm, more preferably at most 1 cm.

M2. Method for storing a plurality of containers at different vertical levels within a processing device, the method comprising

storing the containers such that a vertically protruding portion of at least one of the plurality of containers vertically overlaps and/or extends beyond a container at an adjacent level being higher than the level on which the at least one of the plurality of containers is stored.

M3. The method according to the preceding method embodiment, wherein the method further comprises the features of M1.

M4. Method for storing a plurality of containers at different vertical levels within a processing device, the method comprising

storing the containers with a horizontal offset between adjacent vertical levels.

M5. The method according to the preceding method embodiment, wherein the method further comprises the features of M1, M2 and/or M3.

M6. The method according to any of the preceding method embodiments, wherein the method comprises storing the plurality of containers in an inlay system comprising a plurality of support structures, each of which is configured to support at least a portion of at least one container.

M7. The method according to the preceding method embodiment, wherein the inlay system is an inlay system according to any of the preceding system embodiments.

M8. The method according to any of the 2 preceding method embodiments, wherein the method comprises storing the containers such that a vertical distance V between a vertically protruding portion of at least one of the plurality of containers and the support structure of an adjacent level being higher than the level supporting the at least one of the plurality of containers is at most 5 cm, preferably at most 2.5 cm, more preferably at most 1 cm.

M9. The method according to any of the 3 preceding method embodiments, wherein the method comprises storing the containers such that a vertically protruding portion of at least one of the plurality of containers vertically overlaps and/or extends beyond the support structure of an adjacent level being higher than the level supporting the at least one of the plurality of containers.

M10. The method according to any of the 4 preceding method embodiments, wherein the method comprises storing the containers such that neither the support structure adjacent in the direction of the vertically protruding portion of at least one of the plurality of containers nor a container stored upon said adjacent support structure horizontally overlaps with said protruding portion.

M11. The method according to any of the 5 preceding method embodiments, wherein the method comprises storing each of the plurality of containers on a support structure of the inlay system.

M12. The method according to any of the 6 preceding method embodiments, wherein the method comprises installing the inlay system in a processing device such that there is a horizontal offset between support structures of adjacent levels.

M13. The method according to any of the preceding method embodiments with the features of M4 and/or M12, wherein the horizontal offset between adjacent levels is in the same direction for each level.

M14. The method according to any of the preceding method embodiments with the features of M4 and/or M12 and excluding the features of M13, wherein the horizontal offset between adjacent levels alternates in opposite directions.

M15. The method according to any of the preceding method embodiments, wherein the method comprises removing a container from one level without removing the container of the above level.

M16. The method according to any of the preceding method embodiments, wherein the method comprises providing a fluid connection to at least one of the plurality of containers, wherein the fluid connection vertically overlaps and/or extends beyond a container at an adjacent level.

M17. The method according to any of the preceding method embodiments and with the features of M6, wherein the method comprises providing a fluid connection to at least one of the plurality of containers, wherein the fluid connection vertically overlaps and/or extends beyond the support structure of an adjacent level being higher than the level supporting the at least one of the plurality of containers.

M18. The method according to any of the preceding method embodiments, wherein the method further comprises at least partially filling the container with a fluid.

M19. The method according to any of the preceding method embodiments, wherein the method further comprises ventilating the container.

M20. The method according to any of the preceding method embodiments, wherein the method further comprises one or a plurality of inoculating, feeding, harvesting and/or testing of a sample within the container.

M21. The method according to any of the preceding method embodiments, wherein the processing device is a device according to any of the preceding device embodiments.

M22. The method according to any of the preceding method embodiments, wherein the method comprises cultivating cells within the plurality of containers.

Below, reference will be made to use embodiments. These embodiments are abbreviated by the letter “U” followed by a number. Whenever reference is herein made to “use embodiments”, these embodiments are meant.

U1. Use of the inlay system according to any of the preceding system embodiments for storing a plurality of containers at different vertical levels within a processing device.

U2. Use according to the preceding use embodiment, wherein the processing device is a device according to any of the preceding device embodiments.

U3. Use according to any of the 2 preceding use embodiments, wherein the use is for performing the method according to any of the preceding method embodiments.

Embodiments of the present invention will now be described with reference to the accompanying drawings. These embodiments should only exemplify, but not limit, the present invention.

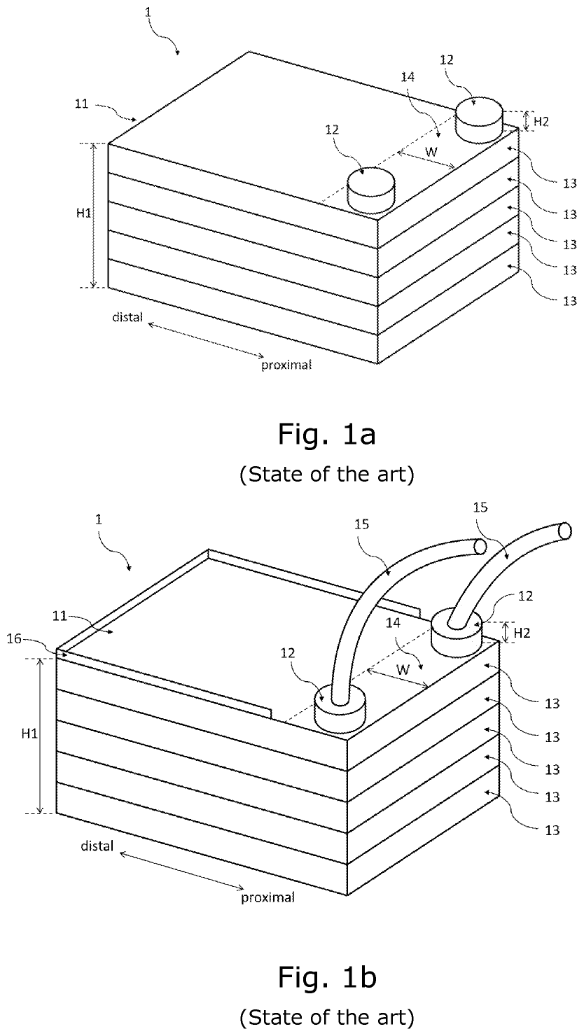

FIGS. 1a and 1b depict a multi-layered container according to the state of the art;

FIGS. 2a to 2d depict embodiments of an inlay system comprising a horizontal offset between adjacent levels;

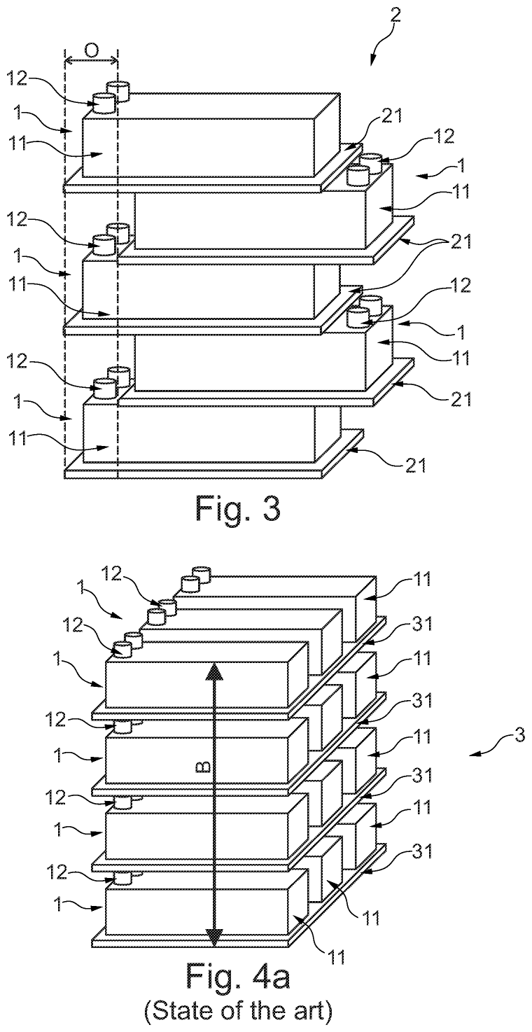

FIG. 3 depicts another embodiment of an inlay system comprising a horizontal offset between adjacent levels;

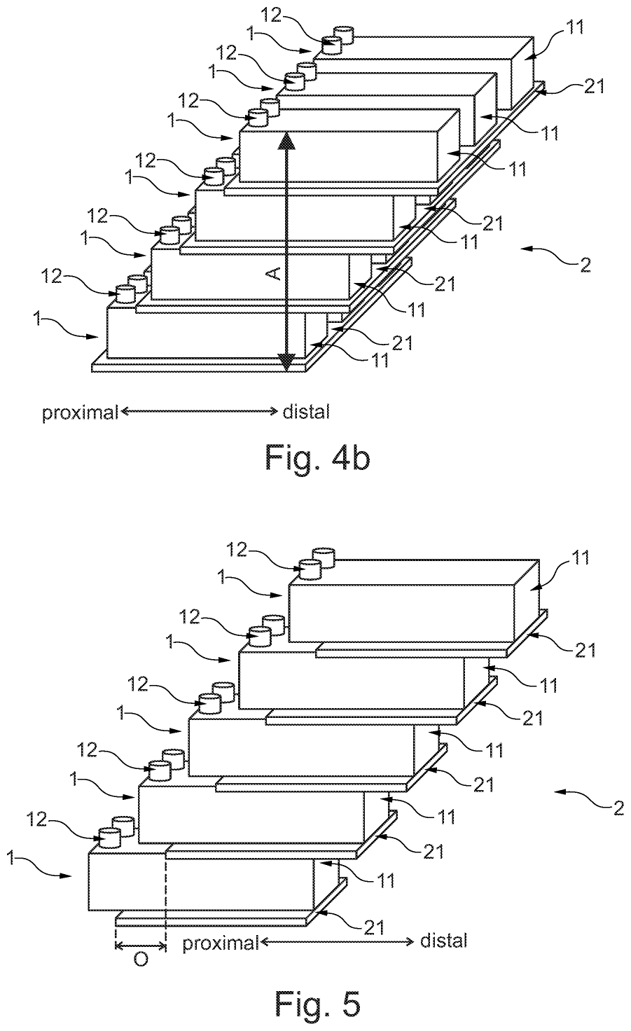

FIGS. 4a and 4b illustrate the improved space efficiency of an inlay system according to the present invention compared to the state of the art;

FIG. 5 depicts a further embodiment of an inlay system comprising a horizontal offset between adjacent levels, wherein the support structures directly support only a portion of a respective container;

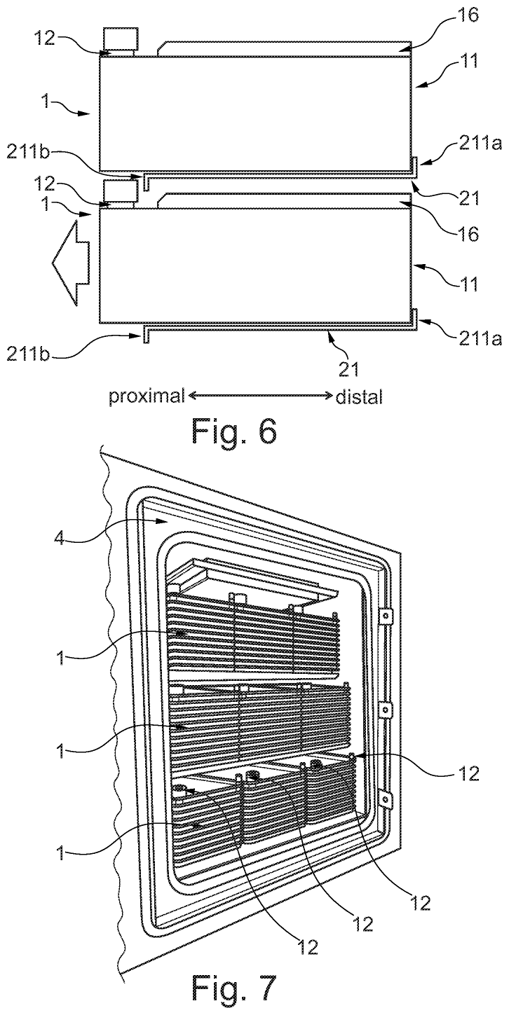

FIG. 6 illustrates an inlay system with support structures comprising stiffening elements; and

FIG. 7 illustrates an inlay system according to the present invention within a processing device.

It is noted that not all the drawings carry all the reference signs. Instead, in some of the drawings, some of the reference signs have been omitted for the sake of brevity and simplicity of the illustration. Embodiments of the present invention will now be described with reference to the accompanying drawings.

With reference to FIGS. 1a and 1b, an exemplary embodiment of a container 1 as known in the art is discussed. As already mentioned, it is known to use multi-layered sample containers 1 for simultaneous processing of samples under identical conditions. Such processing of samples may for example refer to cell cultivation, i.e. growing cells under controlled conditions, and such containers may more generally for example be utilized for the production of cells, vaccines, and therapeutic proteins.

Such a container 1 (also referred to as sample container 1 or multi-layered sample container 1) may generally comprise a main portion 11 constituting the main part of the container that may typically hold any fluids and/or samples, e.g., cells, during processing. When referring to the general contour of the container 1, the contour of the main portion 11 is meant. The main portion 11 may comprise a cube or cuboid shape, wherein one or more corners and/or edges may be rounded.

The main body 11 may comprise one or a plurality of permanently stacked trays 13, which may all be fluidly interconnected, e.g., to allow providing all of them with a fluid through a single tube connected to the container 1. Such a fluid connection may for example be provided through a channel running vertically downwards along and/or within the stacked trays. For example, in one of a (rounded) corner of the main body. The individual trays 13 may for example be permanently interconnected by means of welding or gluing. Generally, such containers may be made from polymer, such as polystyrene. In particular, the container 1 may be made of a transparent material to advantageously allow of easy inspection of the inner volume of the container 1 and its trays 13.

Furthermore, a container 1 may typically comprise at least one protruding portion 12 that extends vertically beyond the main portion 11 and may thus also be referred to as vertically protruding portion 12. Such protruding portions 12 may generally be configured to allow for access to an inner volume of the container 1, e.g., to an inner channel providing access to the trays 13 as discussed above. In other words, the protruding portion 12 may provide a port allowing for easy access to the inner volume of the container 1. With reference to FIG. 1b such a protruding portion 12 may generally be a connector that may allow for connecting further elements, such as a tube 15, a filter element, a sensor, a cap, a cap adaptor, a vent filter, a tube inlet, a connector, an adaptor cap, a port cover or the like, and/or combinations of those. Thus, the protruding portion 11 may provide a fluidic access to the inner volume of the container 1 for various different elements and depending on the respective requirements. Access to the inner volume may for example be used for providing a fluid to the inner volume, providing ventilation of the inner volume, and/or inoculation, feeding, harvesting and/or testing of a sample within the container.

Again with reference to FIG. 1b, the container 1 may further comprise a rim 16 vertically extending from the main portion 11 of the container 1. The rim 16 may run around at least a portion of a circumference of the main portion. Preferably the rim 16 may vertically extend in the same direction as the at least one protruding portion 12. Generally, the rim 16 may comprise a rim height that is smaller than the protruding portion height H2. In other words, the rim extends less in the vertical direction than the at least one protruding portion 12.

The main portion 11 of such a container may comprise a main portion height H1, which denotes the height of the main portion and thus does not include the vertically protruding portion 12. The main portion height H1 may generally depend on the number of layers/trays of the multi-layer container 1. Furthermore, the vertically protruding portion may comprise a protruding portion height H2 denoting the height, i.e. the vertical extension, of the at least one protruding portion. A total height of the container may thus amount to H1+H2.

The at least one protruding portion 12 may typically be located in a proximal section 14 of the container 1.

That is, the remaining part of the container 1 may be more distal than the proximal section 14 comprising the at least one protruding portion 12. Typically, the proximal section 14 may comprise less volume than the remaining part of the container 1. In other words, the proximal section 14 of the container 1 is more proximal than the remaining part of the container in a horizontal direction. The proximal section 14 may comprise a proximal section width W, which may denote a horizontal extend of the proximal section 14. Further, the rim 16 may preferably not extend into the proximal section of the container. That is, the rim 16 may preferably only run along the circumference of the main portion 11 that is not comprised by the proximal section 14.

It will be understood that while the schematic illustration of the container in FIGS. 1a and 1b shows a container 1 comprising five trays 13, i.e. a container comprising five layers, a container may generally comprise a different number of trays/layers, e.g., 1, 2, 4, 5, 10, 20, or 40. Furthermore, in an inlay system according to the present invention containers with different numbers of layers/trays may be stored. That is, the plurality of containers stored in an inlay system according to the present invention may not all comprise the same number of trays/levels.

For storing, i.e. placing, a plurality of such containers 1 in a processing device it is known to use an inlay system that allows to store said containers 1 at different vertical levels. However, in known inlay systems, the containers are arranged directly vertically on top of each other, which is disadvantageously not very space efficient (cf. FIG. 4). Furthermore, this either does not allow to connect any tubing or other elements to the protruding portion 12, which provides a port and/or access to the inner volume of the container 1, or it requires significant spacing of adjacent levels to not only accommodate the protruding portions 12 but also the tubing 15 or other elements connected to the protruding portions 12.

The present invention therefore provides alternative embodiments of an inlay system 2 for storage of a plurality of containers 1 at different vertical levels within a processing device. The inlay system 2 comprises a plurality of support structures 21, wherein each of said support structures is configured to support at least a portion of at least one container 1.

With respect to FIGS. 2a to 2d, the support structures 21 of the inlay system 2 according to an embodiment of the present invention are arranged with a horizontal offset O between adjacent levels. In other words, the support structure 21 of an adjacent level is shifted by a horizontal offset O. In the depicted embodiment, the horizontal offset O is in the same direction for each level. That is, starting at the bottom (lowest container in FIG. 2), each adjacent level is shifted in a distal direction by a horizontal offset O. That is, the support structure 21 and the respective supported container 1 is shifted by a horizontal offset O in the distal direction. In other words, the support structures 21 are arranged in consecutive levels, wherein each level is shifted by a horizontal offset O with respect to the preceding level. While FIGS. 2a and 2b depict an inlay system 2 wherein the horizontal offset O is identical between all pairs of adjacent levels, it will be understood that this need not be the case. Instead, the horizontal offset O may be different between some or all of the adjacent levels. For example, with reference to FIG. 2c the horizontal offset O, O1, O2 between different pairs of adjacent levels may be different. In the embodiment depicted in FIG. 2c the horizontal offset O1 between the lower two levels/support structures 21, 21a, 21b is for example smaller than the horizontal offset O2 between the upper two levels/support structures 21, 21b, 21c.

Furthermore, it will be understood that the support structures 21, 21a, 21b, 21c do not necessarily comprise the same support structure length L, La, Lb, Lc, but that instead an inlay system 2 may comprise support structures 21, 21a, 21b, 21c comprising different support structure lengths La, Lb, Lc, as depicted in FIG. 2c. It will be understood that support structure length L, La, Lb, Lc denotes the extension of the support structure 21, 21a, 21b, 21c in horizontal direction, particularly in a proximal to distal direction. In other words, the support structure length L, La, Lb, Lc may denote the extension of a respective support structure 21, 21a, 21b, 21c in a direction running from an opening of the processing device in which the inlay system 2 is installed to a backside of the inner volume, i.e. the side of the inner volume lying opposite to the opening through which the inner volume of the processing device may be accessed. If the inlay system comprises a horizontal offset O, O1, O2, the direction corresponds to the direction in which the support structures are horizontally offset. In some embodiments, each of the plurality of support structures may comprise a different support structure length.

Generally, the horizontal offset O may be chosen such that neither the support structure 21 adjacent in the direction of the vertically protruding portion 12 of at least one of the plurality of containers 1 nor a container 1 stored upon said adjacent support structure 21 horizontally overlaps with said protruding portion. For example, in the depicted embodiment, the vertically protruding portions 12 extend in an upward direction and the horizontal offset O is configured such that the protruding portions 12 do not horizontally overlap with the support structure 21 and the container 1 of the adjacent above level.

Such an arrangement advantageously enables the vertically protruding portion 12 of a container to vertically overlap and/or extend beyond the support structure 21 of an adjacent level. Additionally or alternatively, such an arrangement advantageously allows for the connection of tubes and/or other elements to the containers 1, as indicated in FIG. 2b. Particularly, it may allow for connecting tubes 15 and/or other elements to the protruding portions 12 of the containers 1, wherein said connected elements can overlap and/or extend beyond the support structure 21 of an adjacent level, therefore allowing for an improved efficiency in terms of usage of the vertical space available in a processing device. In this regard, other elements may for example refer to a filter element, a sensor, a cap, a cap adaptor, a vent filter, a tube inlet, a connector, an adaptor cap, a port cover or the like and/or combinations of those. In order to accommodate such connections without the horizontal offset O significantly more space would be required, which could not be used for storage of containers 1. That is, through introduction of the horizontal offset, the volume within a processing device actually occupied by containers 1 may advantageously be increased compared to standard vertical stacking of containers, while maintaining the possibility to connect tubes and/or other elements to each of the containers.

It is noted that the FIGS. 2a to 2c depict inlay systems 2 wherein the vertical spacing is such that the protruding portion vertically overlaps and/or extends beyond the support structure 21 of the adjacent level. With respect to FIG. 2d, the present invention is also directed to inlay systems 2 wherein the vertical spacing between a lower support structure 21, 21a and a higher support structure 21, 21b being adjacent to the lower support structure 21, 21a is configured so that a vertical distance V between a vertically protruding portion 12, 12a of at least one of the plurality of containers 1, 1a located on the lower support structure 21, 21a and the higher support structure 21, 21b is limited to for example 5 cm, 2.5 cm or 1 cm. That is, the vertical distance V may also be smaller than that. Such an inlay system 2 still advantageously allows for connecting tubes 15 and/or other elements, which would not have been possible in inlay systems known in the art and thus allows for a more space efficient storage of the containers.

Generally, it will be understood that support structure 21, 21a and/or container 1a that is closest to the bottom will be considered to be the lowest support structure 21, 21a and/or container 1a, such that said support structure 21, 21a and/or container 1a may be referred to as being below/downwards of any of the other support structures 21 and/or containers 1. That is, generally the direction for below/downwards and above/upwards are defined in a stacking direction, e.g., typically with respect to gravity.

Furthermore, it will be understood that the term “adjacent level” and/or “adjacent support structure” refers to a level/support structure that is adjacent in the vertical direction. That is a level/support structure that is directly below or above the current level/support structure.

In an alternate embodiment as depicted in FIG. 3, the direction of the horizontal offset O may alternate between adjacent levels. In other words, the direction of the horizontal offset O between adjacent levels alternates in opposite directions. In such an embodiment also the orientation of the containers alternates between adjacent levels, such that the at least one protruding portion 12 and/or any element attached thereto can vertically overlap and/or extend beyond the support structure 21 and/or the main body 11 of a container 1 of an adjacent level, e.g., a level above. Again, it will be understood that a vertical overlap between the at least one protruding portion 12, 12a and the support structure 21, 21b of an adjacent (higher) level is not necessary for all embodiments of the present invention. Instead some embodiments may provide for a small vertical distance V between the at least one protruding portion 12, 12a and the support structure 21, 21b of an adjacent (higher) level, which may be smaller than known in the state of the art and for example limited to at most 5 cm, preferably limited to at most 2.5 cm, more preferably at most 1 cm.

Again, also for embodiments as depicted in FIG. 3 the horizontal offset O between adjacent levels is such that neither the support structure 21 adjacent in the direction of the vertically protruding portion 12 of at least one of the plurality of containers 1 (here in the upwards direction) nor a container 1 stored upon said adjacent support structure horizontally overlaps with said protruding portion. Therefore, also such an arrangement allows for accessing the containers 1, particularly the protruding portions 12 and thus the inner volume of the containers 1 while providing a more space efficient storage compared to known inlay systems simply providing a vertical stacking of the containers in different levels.

With respect to FIG. 4 an advantage of the present invention is illustrated with respect to inlay system as commonly used. FIG. 4a depicts an inlay system 3 according to the current state of the art, wherein a plurality of containers 1 is stacked at different vertical levels. The inlay system 3 comprises support structures 31 that are arranged at different vertical levels and spaced such that the containers 1 fit between adjacent support structures 31. That is, the spacing of adjacent support structures 31 is such that it accommodates the main portion 11 and the protruding portion 12 of the containers. However, such an arrangement does not allow for connection of any elements to the protruding portions 12 and thus prevents any access to the inner volume of the containers 1. Alternatively, the spacing of adjacent levels may be increased further to allow for such access or respectively connection to the protruding portions, however such an arrangement would lead to a significantly higher space requirement and thus lower storage capacity.

In contrast, FIG. 4b depicts an inlay system 2 according to an embodiment of the present invention, wherein the support structures 21 are arranged with a horizontal offset O between adjacent levels similar to the inlay system 2 depicted in FIG. 2a with the difference that in the embodiment depicted in FIG. 4b each support structure 21 supports a plurality of containers 1.

The comparison of FIG. 4a to FIG. 4b illustrates that a total height B of the inlay system 3 as known in the prior art including the comprised containers 1 is higher than a total height A of the inlay system 2 according to the present invention including the comprised containers. The difference in height is due to the fact that the inlay system 2 according to the present invention allows the protruding portions 12 of the containers to overlap and/or extend beyond the adjacent support structure 21 and/or container 1. This comparison illustrates advantages of embodiments of the present invention, which reduces the overall height required for storage of the same number of containers 1 compared to known systems and further enables easy access to the protruding portions 12 of the containers 1 and/or the inner volume of the container.

With reference to FIG. 5, the support structures 21 may only directly support a portion of the one or more containers 1 stored thereon. That is, the support structure 21 may not run underneath the whole container, but only under a portion thereof. In general, the support structure may support more than 50% of the footprint of the container, but less than, e.g., 95% of the footprint of the container. Preferably, the non-supported portion may be located at the portion of the container 1 comprising the at least one protruding portion. In some cases, the non-supported portion may preferably comprise the proximal section 14 of the container 1 comprising the at least one protruding portion 12. In other words, the support structure 21 (e.g., a bent sheet metal part or a wire mesh) does not have to support the container 1 over its entire surface, but can be arranged only in areas under the container (cf. FIGS. 5 and 6). This may advantageously facilitate the handling (e.g., easier gripping) of the container.

The support structures 21 may be made of metal, preferably a sheet metal or a wire mesh. The sheet metal may be a bent sheet metal. Furthermore, at least one (e.g., all) of the support structures 21 may comprise at least one stiffening element. The at least one stiffening element may for example comprise at least one of a bend, an offset, a hem (e.g., a closed hem), a hole, a slot, a seam (e.g., a standing seam), a tab, a notch, a flange, an emboss, a dimple and/or a rib. Generally, such stiffening elements may be provided through bending, flanging, hemming and/or embossing. Additionally or alternatively, the at least one stiffening element may comprise a bracket or a plate, such as a sheet metal flap.

Generally, the arrangement and/or vertical extension of the at least one stiffening element 211 may be such that it does not obstruct removing a container 1 on a level below the support structure 21. In particular, a stiffening element 211 that extends downwards, i.e. vertically in the direction of a level below the support structure 21, may be located distal of the respective proximal section of a container 1 stored in the level below the respective stiffening element 211. An exemplary embodiment of a support structure 21 comprising two stiffening elements 211, 211a, 211b is depicted in FIG. 6. The support structure 21 comprises two stiffening elements 211, 221a, 221b, in the depicted embodiment for example sheet metal flaps or standing seams. In particular, one of the stiffening elements 211a is located distal of the supported container 1 and extends in an upward direction, i.e. in a direction towards the above level. This may advantageously facilitate handling of the container within the inlay system 2 and/or provide a boundary to stop the container 1 from moving further in the distal direction. The other stiffening element 211b in this embodiment extends downwards, i.e. in a direction towards the below level. This stiffening element 211b is located such that it does not obstruct removing of the below container 1 as indicated by the arrow. This is achieved by the stiffening element 211b being located distal of the protruding portion of the below container, or in other words distal of the proximal section 14 of the below container.

Put differently, stiffening elements 211 (such as sheet metal plates) can be placed in zones that are “behind” protruding portions 12 and/or protruding elements of the container on the level below (e.g., elements connected to the container). The stiffening elements may be kept short enough so that it is still possible to pull out the container stored underneath (FIG. 6).

Again, such stiffening elements 211, 211a, 211b generally provide additional stiffness to the respective support structure 21. For example, a stiffening element 211, 211a, 211b may reduce and preferably prevent the support structure 21 bending down owing to the weight of containers stored thereon. Thus, a stiffening element 211, 211a, 211b may advantageously improve the rigidity of the support structure.

Also, such an arrangement allows for the protruding portion 12 to vertically overlap the support structure 21 of an adjacent level (i.e., the highest point of the protruding portion 12 may be located above the lowest section of the support structure 21), while still enabling easy access to and handling of the containers 1 and rendering the support structure more rigid, particularly with respect to bending of the support structure under load. Therefore, also the embodiment of the inlay system 2 depicted in FIG. 6 allows for a more space efficient stacking than for inlay systems 3 as known in the prior art, since the protruding portion 12 vertically overlaps with the support structure 21 of an adjacent level, here, the level above. However, it will be understood that the support structure 21 depicted in FIG. 6 may also be combined with a horizontal offset as for example depicted in FIG. 2.

FIG. 7 shows a plurality of containers stored, i.e. placed, in a processing device 4, e.g., an incubator, by means of an inlay system 2. In other words, the inlay system 2 is installed within an inner volume of the processing device 4, which may be accessed through an opening, such as a door. The inner volume may for example be a chamber. The processing device may be configured for processing of samples within its inner volume/chamber, e.g., samples in containers 1 stored on a respective inlay system 2. The processing device may be configured to control a parameter within the inner volume/chamber and/or the containers stored therein, preferably an environmental parameter such as temperature, humidity, concentration of gases like O₂ and/or CO₂. In the depicted configuration nine containers 1 are stored in three different levels, wherein each level provides room for three containers 1. In such a processing device 4, the containers 1 may be stored in a respective inlay system 2 according to embodiments of the present invention, wherein the support structures 21 are arranged with a horizontal offset between adjacent levels. That is, starting from the lowest level, each adjacent level is horizontally offset in a distal direction, i.e. in a direction into the inner volume of the processing device 4 and away from a front opening of the processing device.

Overall, the present invention thus provides for an inlay system 2 and/or storage method that allows for space-efficient storage of sample containers in processing devices, e.g., heating cabinets. That is, the present invention advantageously provides for space-efficient arrangement of sample containers in the interior of a processing device such as a heating cabinet (e.g., a CO₂ cell incubator), which may advantageously allow to increase the storage capacity of a given processing device. Very generally, the present invention provides an inlay system and/or storage method wherein the distance between adjacent levels can be reduced. In preferred embodiments the inlay system 2 and/or storage method provides for a vertical container arrangement comprising a horizontal offset between adjacent levels. This horizontal offset may provide space in which connections and/or attachments can be accommodated without requiring a correspondingly large distance between adjacent levels. Furthermore, the distance between the levels may be reduced, as elements with the highest vertical space requirement are placed next to the support structure and/or container of the next (adjacent) level.

Thus, containers 1 which, due to the application, are stored in a processing device, (e.g., heating cabinet) with tube accesses, tube connections, filter elements or sensors, can advantageously be arranged more space efficiently in the inner receptacle of the processing device. In fact, the space-efficient arrangement according to the present invention was tested on heating cabinets. Significantly higher packing densities (greater than +25%) were shown when arranging sample containers with elements at the container connections

It is noted that the term processing device or simply device may for example refer to a device providing a controlled environment, e.g., a fixed temperature, humidity, CO₂-Level and/or O₂-Level. Such a device may thus for example be a heating cabinet, an incubator or the like. That is, processing device may generally refer to a device for processing, treatment and/or storage of containers and respective samples comprised by the containers.

Furthermore, the term sample may refer to any kind of sample processed in such a device, in particular sample may also refer to cell cultures processed in a respective container. Furthermore, processing a sample may refer to any step necessary for cultivating, growing maintaining, testing of the sample, or the like. For example, processing may include inoculating, providing fluids to an inner volume of a container, providing ventilation, feeding of the sample (e.g., cells), harvesting, etc.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

While in the above, a preferred embodiment has been described with reference to the accompanying drawings, the skilled person will understand that this embodiment was provided for illustrative purpose only and should by no means be construed to limit the scope of the present invention, which is defined by the claims.

What is claimed:

1. An inlay system for storage of a plurality of containers at different vertical levels within a processing device, the inlay system comprising: a plurality of support structures, each of which is arranged at a different vertical level and configured to respectively support at least a portion of at least one of the plurality of containers, wherein the support structures are arranged with a horizontal offset (“O”) between adjacent levels.

2. The inlay system according to claim 1, wherein a vertical spacing between a lower support structure and a higher support structure being adjacent to the lower support structure is configured so that a vertical distance (“V”) between a vertically protruding portion of at least one of the plurality of containers located on the lower support structure and the higher support structure is at most 5 cm.

3. The inlay system according to claim 1, wherein a vertical spacing between a lower support structure and a higher support structure being adjacent to the lower support structure is configured so that a vertically protruding portion of at least one of the plurality of containers located on the lower support structure vertically overlaps and/or extends beyond the higher support structure.

4. The inlay system according to claim 1, wherein the horizontal offset is such that neither the support structure adjacent in the direction of the vertically protruding portion of at least one of the plurality of containers nor a container stored upon said adjacent support structure horizontally overlaps with said protruding portion.

5. The inlay system according to claim 1, wherein at least one of the support structures comprises at least one stiffening element, wherein the at least one stiffening element comprises at least one of a bend, an offset, a hem, a hole, a slot, a seam, a tab, a notch, a flange, an emboss, a dimple and/or a rib.

6. The inlay system according to claim 1, wherein the horizontal offset O is in the range of 2 to 10 cm.

7. The inlay system according to claim 1, wherein each of the plurality of containers comprises a main portion and at least one of the plurality of containers comprises at least one protruding portion that extends vertically beyond the main portion of the respective container.

8. The inlay system according to claim 7, wherein the main portion comprises main portion height H1 and the at least one protruding portion of the at least one container comprises a protruding portion height H2, wherein for at least one container comprising a protruding portion and being stored on a lower support structure a vertical distance H3 between the lower support structure and a higher support structure being adjacent to the lower support structure is chosen such that H1<H3<(H1+H2).

9. The inlay system according to claim 1, wherein the processing device is one of a heated and/or refrigerated incubator, an environmental chamber, a heating cabinet or a cooling cabinet.

10. The inlay system according to claim 1, wherein a higher support structure being adjacent to a lower support structure is configured such that a vertically protruding portion of at least one of the plurality of containers located on the lower support structure does not horizontally overlap with the respective higher support structure.

11. An inlay system for storage of a plurality of containers at different vertical levels within a processing device, the inlay system comprising: a plurality of support structures, each of which is arranged at a different vertical level and configured to respectively support at least a portion of at least one of the plurality of containers, wherein a vertical spacing between a lower support structure and a higher support structure being adjacent to the lower support structure is configured so that a vertical distance V between a vertically protruding portion of at least one of the plurality of containers located on the lower support structure and the higher support structure is at most 5 cm.

12. A device for processing samples, the device comprising an inlay system for storage of a plurality of containers at different vertical levels within a processing device, the inlay system comprising a plurality of support structures, each of which is arranged at a different vertical level and configured to respectively support at least a portion of at least one of the plurality of containers, wherein the support structures are arranged with a horizontal offset between adjacent levels.

13. The device according to claim 12, wherein the device is configured to control at least one or a plurality of, temperature, humidity, CO₂-level, and/or O₂-level within the device and/or containers stored therein.

14. A method for storing a plurality of containers at different vertical levels within a processing device, the method comprising:
storing the containers with a horizontal offset between adjacent vertical levels.

15. The method according to claim 14, wherein the method comprises storing the plurality of containers in an inlay system comprising a plurality of support structures, each of which is configured to support at least a portion of at least one container.

16. The method according to claim 15, wherein the method comprises storing the plurality of containers such that a vertical distance V between a vertically protruding portion of at least one of the plurality of containers and the support structure of an adjacent vertical level being higher than the vertical level supporting the at least one of the plurality of containers is at most 5 cm.

17. The method according to claim 15, wherein the method comprises storing the plurality of containers such that a vertically protruding portion of at least one of the plurality of containers vertically overlaps and/or extends beyond the support structure of an adjacent level being higher than the level supporting the at least one of the plurality of containers.

18. The method according to claim 15, wherein the method comprises installing the inlay system in a processing device such that there is a horizontal offset between support structures of adjacent levels.

19. The method according to claim 15, wherein the method comprises storing each of the plurality of containers on a support structure of the inlay system.

20. The method according to claim 14, wherein the method comprises removing a container from one level without removing the container of the above level.
